(19) United States
(12) Patent Application Publication
(10) Pub. No.: US 2008/0233199 A1
(43) Pub. Date: Sep. 25, 2008

(54) Coacervation Process

(75) Inventors: Rajesh Kumar, Marlborough, MA (US); Gregory Troiano, Pembroke, MA (US); J. Michael Ramstack, Lunenburg, MA (US); Paul Herbert, Wayland, MA (US); Michael Figa, Allston, MA (US)

Correspondence Address: ELMORE PATENT LAW GROUP, PC, 515 Groton Road, Unit 1R, Westford, MA, 01886, US

(73) Assignee: Alkermes, Inc., Cambridge, MA (US)

(21) Appl. No.: 12/051,395

(22) Filed: Mar. 19, 2008

Related U.S. Application Data
(60) Provisional application No. 60/919,378, filed on Mar. 22, 2007.

(51) Int. Cl. A61K 9/16; A61K 38/00

(52) U.S. Cl. 424/499; 424/489; 514/2

(57) ABSTRACT

Methods of forming compositions for the sustained release of water soluble active agents, including biologically active polypeptides and products produced by the process are described. Improved product characteristics and ease of scale-up can be achieved using a novel coacervation process wherein at least one coacervation agent is added to the mixture comprising the active agent and the polymer in at least two distinct stages.

### RELATED APPLICATION

This application claims the benefit of U.S. Provisional Application No. 60/919,378, filed on Mar. 22, 2007. The entire teachings of the above application are incorporated herein by reference.

### BACKGROUND OF THE INVENTION

Numerous proteins and peptides, collectively referred to herein as polypeptides, exhibit biological activity in vivo and are useful as medicaments. Many illnesses or conditions require maintenance of a sustained level of medicament to provide the most effective prophylactic and/or therapeutic effects. Sustained levels are often achieved by the administration of biologically active polypeptides by frequent subcutaneous injections, which often results in fluctuating levels of medicament and poor patient compliance.

As an alternative, the use of biodegradable materials, such as polymers, encapsulating the medicament can be employed as a sustained delivery system. The use of biodegradable polymers, for example, in the form of microparticles or microcarriers, can provide a sustained release of medicament, by utilizing the inherent biodegradability of the polymer to control the release of the medicament thereby providing a more consistent, sustained level of medicament and improved patient compliance.

A variety of methods is known by which compounds can be encapsulated in the form of microparticles. In these methods, the material to be encapsulated (drugs or other active agents) is generally dissolved, dispersed, or emulsified, using stirrers, agitators, or other dynamic mixing techniques, in a solvent containing the matrix forming material. Solvent is then removed from the microparticles and thereafter the microparticle product is obtained.

Many of the published procedures for microencapsulation with biodegradable polymers employ solvent evaporation/extraction techniques, wherein an oil phase comprising the active agent and the polymer in an organic solvent is dispersed in a continuous aqueous phase. The solvent diffuses out of the oil phase droplets, resulting in the formation of microparticles. These techniques are particularly suitable for water insoluble drugs because these drugs will tend not to partition into the continuous aqueous phase. On the other hand, water soluble drugs may partially partition into the aqueous phase during the preparation process, resulting in a low encapsulation efficiency.

Non-solvent induced coacervation or phase separation, referred to herein as coacervation, is a method which has frequently been employed to prepare microparticles comprised of a biodegradable polymeric matrix and a water soluble biologically active agent. The coacervation method utilizes a continuous phase that is a non-solvent for the polymer and in which hydrophilic active agents also are not soluble. Drug partitioning into the continuous phase does not occur to an appreciable extent, and relatively high encapsulation efficiencies are typical.

In a conventional coacervation process, a known amount of polymer, such as poly-(lactide-co-glycolide), PLG, with a monomeric molar ratio of lactide to glycolide ranging from 100:0 to 50:50, is dissolved in an appropriate organic solvent. A solid drug, preferably lyophilized and micronized, may be dispersed in the polymer solution, where it is insoluble or slightly soluble in the organic solvent. Alternatively, the active agent may be dissolved in water, or in water which contains some additives, and emulsified in the polymer solution, forming a water-in-oil emulsion. The resultant suspension or emulsion is then added to a reactor and addition of a non-solvent, or coacervation agent, is initiated at a predetermined rate. Addition of the coacervation agent results in the formation of a dispersion of coacervate droplets containing polymer, active agent and polymer solvent. The coacervate droplets are also referred to as nascent microparticles or embryonic microparticles. At the completion of the coacervation agent addition, the coacervate is transferred into a quench liquid containing a hardening agent to solidify the semi-solid microparticles. The hardened microparticles are collected, washed, and dried to remove solvents to a suitable level.

The coacervation process generally provides good encapsulation efficiency for water-soluble active agents and can be optimized to produce microparticles that are acceptable with respect to critical attributes including particle size distribution, residual solvent levels, and the time course of drug release in vitro or after injection into a patient. However, the coacervation technique is not easily converted into a process for producing commercial scale quantities of microparticles because processing parameters, e.g., rate of non-solvent addition, agitation conditions, and the viscosity of both the active agent/polymer mixture and the coacervation agent must be empirically optimized by trial and error at each stage of scale-up. Thus, scale-up of conventional coacervation processes is not only time consuming, but imprecise. Furthermore, large-scale production of microparticles by coacervation requires the storage, use and eventual disposal of large quantities of organic solvents, such as heptane, employed in the hardening of the microparticles.

US Patent Application No. 20060110423, incorporated herein by reference, discloses compositions for the sustained release of biologically active polypeptides, and methods of forming and using said compositions, for the sustained release of biologically active polypeptides. The sustained release compositions comprise a biocompatible polymer, and agent, such as a biologically active polypeptide, and a sugar. The agent and sugar are dispersed in the biocompatible polymer separately or, preferably, together. In a particular embodiment, the sustained release composition is characterized by a release profile having a ratio of maximum serum concentration (Cₘₐₓ) to average serum concentration (Cₐᵥₑ) of about 3 or less.

The aforementioned US patent application discloses a process for forming a composition for the sustained release of biologically active polypeptide. In this process, an aqueous phase comprising water, a water soluble polypeptide and a sugar, is combined with an oil phase comprising a biocompatible polymer and a solvent for the polymer, forming a water-in-oil emulsion. A coacervation agent, for example silicone oil, vegetable oil or mineral oil, is added to the mixture to form embryonic microparticles; which are subsequently transferred to a quench solvent to undergo hardening. The hardened microparticles are then collected and dried. The disclosed process is conducted in a batch mode using stirred tank reactors and ranges in scale from 100 gram to 1 kg. While this process can yield microparticles with suitable characteristics of particle size, residual solvents and drug release kinetics, it suffers from the aforementioned need for time-consuming and expensive development work to establish empirically-determined scale-up parameters, and requires consumption and disposal of large volumes of organic solvents.

One approach to aiding the scale-up process is to use a static mixer to form the microparticles, as disclosed in U.S. Pat. No. 5,654,008, incorporated herein by reference. In the method disclosed in U.S. Pat. No. 5,654,008, a first phase, comprising the active agent and the polymer, and a second phase are pumped through a static mixer into a quench liquid to form microparticles containing the active agent. U.S. Pat. No. 5,654,008 further describes an embodiment of this method wherein microparticles are formed by coacervation. In this case, the first phase is a dispersion of water-soluble drug, which is either in the form of a micronized solid or an aqueous solution, in a solution of PLG in a solvent. The second phase is silicone oil, a coacervation agent; and the quench liquid is heptane. The drug-PLG dispersion and the silicone oil are pumped through a static mixer and the outflow is directed into a quench tank containing heptane. The semi-solid microparticles are hardened, collected by vacuum filtration, washed with fresh heptane and then dried under vacuum. While this process produces microparticles and is inherently more scaleable than the corresponding batch process, the resultant particle size distribution is broad. Moreover, the organic solvent consumption is high because the hardening step is performed in batch mode.

Thus, a need exists for a coacervation process that produces a consistent particle size, residual solvent and drug release profile, is readily scaleable to process scale, and is efficient with respect to consumption of process solvents.

### SUMMARY OF THE INVENTION

This invention relates to methods of forming compositions for the sustained release of water soluble active agents, including biologically active polypeptides. The invention further relates to the discovery that improved product characteristics and ease of scale-up can be achieved using a novel coacervation process wherein at least one coacervation agent is added to the mixture comprising the active agent and the polymer in at least two distinct stages.

One aspect of the invention is a method for preparing microparticles comprising:

(a) providing a first phase comprising an active agent, a biocompatible polymer and a solvent;

(b) forming a coacervate; and

(c) combining the coacervate with a quench liquid, thereby forming microparticles containing the active agent;

wherein step (b) comprises

(i) adding a first coacervation agent to the first phase; and subsequently

(ii) adding a second coacervation agent.

This aspect of the invention includes a method for forming compositions for the sustained release of biologically active agents, such as polypeptides, which comprises forming a first phase comprising the active agent, a polymer and a solvent; adding coacervation agent to the mixture in at least two sequential stages to form embryonic microparticles; transferring the embryonic microparticles to a quench liquid to harden the microparticles; collecting the hardened microparticles; and drying the microparticles. The first phase can be a water-in-oil emulsion prepared by dispersing by, for example, sonication or homogenization, an aqueous solution of the active agent in an organic solution comprising a biocompatible polymer and a solvent for the polymer. When the first phase is a water-in-oil emulsion, it can also be referred to as the inner emulsion or the primary emulsion. Alternatively, the first phase can be a suspension wherein the drug in the solid state is dispersed in an organic solution comprising a biocompatible polymer and a solvent for the polymer.

In a particular embodiment, the solvent is methylene chloride and the coacervation agent is silicone oil. The coacervation agent is added in two or more stages. That is, the second stage must be distinct and separate from the first stage and not a mere continuous extension of it. In the first stage, the coacervation agent is added in an amount sufficient to achieve a coacervation agent to polymer solvent ratio of less than 1:1, preferably from about 0.3:1 to about 0.5:1. In a subsequent stage, additional coacervation agent is added in an amount sufficient to achieve a final coacervation agent to polymer solvent ratio of at least 1:1, preferably between 1:1 and 3:1, more preferably about 1.5:1. The rate and manner in which the coacervation agent is added during each stage can be optimized in order to yield microparticles of suitable particle size distribution, residual solvent levels and drug release kinetics.

Additionally or alternatively, the invention includes a method of forming compositions for the sustained release of biologically active agents, such as polypeptides, which comprises forming a first phase comprising the active agent, a polymer and a solvent; adding coacervation agent to the mixture in at least two sequential stages to form embryonic microparticles, wherein the first stage of addition takes place in a stirred tank reactor; transferring the embryonic microparticles to a quench solvent to harden the microparticles; collecting the hardened microparticles; and drying the hardened microparticles. In a particular embodiment, the first addition of coacervation agent to the mixture comprising the active agent and the polymer in a stirred tank reactor is through a plurality of addition ports, thereby facilitating efficient mixing of the coacervation agent with the active agent-polymer mixture. In another embodiment, the first addition of coacervation agent to the mixture comprising the active agent and the polymer in a stirred tank reactor is through at least one spray nozzle, also facilitating efficient mixing of the coacervation agent with the active agent-polymer mixture. In yet another embodiment, the first addition of coacervation agent to the mixture comprising the active agent and the polymer in a stirred tank reactor is performed at a rate which results in completion of the first addition stage after at least about 2 minutes, preferably at least about 3 minutes, and more preferably at least about 5 minutes. The combination of a slow addition of the first coacervation agent, together with efficient blending of the coacervation agent with the mixture comprising the active agent and the polymer, results in a minimal entrapment of coacervation agent in the embryonic microparticles and low residual levels of coacervation agent in the final product. Thus, for example, in the case where the coacervation agent is silicone oil, residual silicon levels of less than 1000 ppm, preferably less than 500 ppm, and more preferably less than 200 ppm, by weight can be achieved.

Additionally or alternatively, the invention includes a method of forming compositions for the sustained release of biologically active agents, such as polypeptides, which comprises forming a first phase comprising the active agent, a polymer and a solvent; adding coacervation agent to the mixture in at least two sequential stages to form embryonic microparticles, wherein at least one stage of addition takes place in a static mixer; transferring the embryonic microparticles to a quench solvent to harden the microparticles; collecting the hardened microparticles; and drying the hardened microparticles. In a particular embodiment, the first coacervation agent addition is made to take place in a static mixer. In preferred embodiment, the final addition of coacervation agent is made to take place in a static mixer. In yet another embodiment, the final addition of coacervation agent is made to take place in a static mixer and the resulting mixture is made to flow through an assembly comprising hollow tubing to provide residence time and some degree of mixing, and a final static mixer before discharging into a quench tank containing a hardening agent.

Additionally or alternatively, the invention includes a method of forming compositions for the sustained release of biologically active agents, such as polypeptides, which comprises forming a first phase comprising the active agent, a polymer and a solvent; adding a coacervation agent to the first phase to form embryonic microparticles, wherein the coacervation agent addition takes place through at least two inlets of a continuous flow mixing apparatus; transferring the resulting coacervate to a quench solvent to harden the microparticles; collecting the hardened microparticles; and drying the hardened microparticles. In a particular embodiment, the continuous flow mixing apparatus is an agitated plug flow reactor with multiple coacervation agent addition ports. In another embodiment, the continuous flow mixing apparatus is a static mixer assembly with multiple coacervation agent addition ports. In yet another embodiment, the continuous flow mixing apparatus is a static mixer with a porous wall through which the coacervation agent is made to flow. In yet another embodiment, the continuous flow mixing apparatus is a series of at least two continuous stirred tank reactors (CSTRs).

Additionally or alternatively, the invention includes a method of forming compositions for the sustained release of biologically active agents, such as polypeptides, which comprises forming a first phase comprising the active agent, a polymer and a solvent; adding a coacervation agent to the first phase to form embryonic microparticles, combining the resulting coacervate with a hardening agent in a static mixer; collecting the hardened microparticles; and drying the hardened microparticles.

This invention relates to methods for forming compositions for the sustained release of agents, such as biologically active polypeptides. The sustained release compositions of this invention comprise a biocompatible polymer, and an agent, such as a biologically active polypeptide. In a preferred embodiment, the biologically active polypeptide is an antidiabetic or glucoregulatory polypeptide, such as GLP-1, GLP-2, exendin-3, exendin-4 or an analog, derivative or agonist thereof, preferably exendin-4.

The sustained release composition may additionally comprise one or more excipients, including but not limited to salts, sugars, carbohydrates, buffers and surfactants. The excipient is preferably sucrose, mannitol or a combination thereof. A preferred combination includes exendin-4 and sucrose and/or mannitol.

Additionally or alternatively, the sustained release composition consists essentially of or, alternatively consists of, a biocompatible polymer, exendin-4 at a concentration of about 3 to 5% w/w and sucrose at a concentration of about 2% w/w. The biocompatible polymer is preferably a poly-lactide-coglycolide polymer.

The agent or polypeptide, e.g. exendin-4, can be present in the composition described herein at a concentration of about 0.01% to about 10% w/w based on the total weight of the final composition. In addition, the sugar, e.g. sucrose, can be present in a concentration of about 0.01% to about 5% w/w of the final weight of the composition.

The compositions of this invention can be administered to a human, or other animal, by injection, implantation (e.g., subcutaneously, intramuscularly, intraperitoneally, intracranially, and intradermally), administration to mucosal membranes (e.g., intranasally, intravaginally, intrapulmonary or by means of a suppository), or in situ delivery (e.g., by enema or aerosol spray).

When the sustained release composition has incorporated therein a hormone, particularly an anti-diabetic or glucoregulatory peptide, for example, GLP-1, GLP-2, exendin-3, exendin-4 or agonists, analogs or derivatives thereof, the composition is administered in a therapeutically effective amount to treat a patient suffering from diabetes mellitus, impaired glucose tolerance (IGT), obesity, cardiovascular (CV) disorder or any other disorder that can be treated by one of the above polypeptides or derivatives, analogs or agonists thereof.

The use of a sugar in the sustained release compositions of the invention improves the bioavailability of the incorporated biologically active polypeptide, e.g., anti-diabetic or glucoregulatory peptides, and minimizes loss of activity due to instability and/or chemical interactions between the polypeptide and other components contained or used in formulating the sustained release composition, while maintaining an excellent release profile.

The advantages of the sustained release formulations as described herein include increased patient compliance and acceptance by eliminating the need for repetitive administration, increased therapeutic benefit by eliminating fluctuations in active agent concentration in blood levels by providing a desirable release profile, and a potential lowering of the total amount of biologically active polypeptide necessary to provide a therapeutic benefit by reducing these fluctuations.

### BRIEF DESCRIPTION OF THE DRAWINGS

FIG. 1 is a graph depicting the time course of coacervation agent addition for a multistage coacervation process.

FIG. 2 is a diagram of a multistage coacervation process described herein in which multiple static mixers are deployed in series.

FIG. 3 is a diagram of a multistage coacervation process described herein in which multiple CSTRs are deployed in series.

FIG. 4 is a diagram of a multistage coacervation process in which coacervation agent is introduced through multiple inlets of an agitated plug flow reactor.

FIG. 5 is a diagram of a hybrid multistage coacervation process employing a stirred tank and a static mixer.

FIG. 6 is a diagram of a continuous coacervation process in which coacervate formation and quench take place in a static mixer.

FIG. 7 is a diagram of the single stage coacervation process employed in Example 3.

### DETAILED DESCRIPTION OF THE INVENTION

This invention relates to methods of forming compositions for the sustained release of biologically active agents, including water soluble active agents such as polypeptides. The invention further relates to the discovery that improved product characteristics and ease of scale-up can be achieved using novel coacervation processes; including processes wherein at least one coacervation agent is added to the mixture comprising the active agent and the polymer in at least two distinct stages; and including processes wherein at least one stage of coacervation addition, or addition of the hardening agent to the coacervate, utilizes a continuous flow mixing device such as a static mixer.

The Agent

Biologically active polypeptides as used herein collectively refers to biologically active proteins and peptides and the pharmaceutically acceptable salts thereof, which are in their molecular, biologically active form when released in vivo, thereby possessing the desired therapeutic, prophylactic and/or diagnostic properties in vivo. Typically, the polypeptide has a molecular weight between 500 and 200,000 Daltons.

Suitable biologically active polypeptides include, but are not limited to, glucagon, glucagon-like peptides such as, GLP-1, GLP-2 or other GLP analogs, derivatives or agonists of glucagon-like peptides, exendins, such as exendin-3 and exendin-4, derivatives, agonists and analogs thereof, vasoactive intestinal peptide (VIP), immunoglobulins, antibodies, cytokines (e.g., lymphokines, monokines, chemokines), interleukins, macrophage activating factors, interferons, erythropoietin, nucleases, tumor necrosis factor, colony stimulating factors (e.g., G-CSF), insulin, enzymes (e.g., superoxide dismutase, plasminogen activator, etc.), tumor suppressors, blood proteins, hormones and hormone analogs and agonists (e.g., follicle stimulating hormone, growth hormone, adrenocorticotropic hormone, and luteinizing hormone releasing hormone (LHRH)), vaccines (e.g., tumoral, bacterial and viral antigens), antigens, blood coagulation factors, growth factors (NGF and EGF), gastrin, GRH, antibacterial peptides such as defensin, enkephalins, bradykinins, calcitonin and muteins, analogs, truncation, deletion and substitution variants and pharmaceutically acceptable salts of all the foregoing.

Alternatively, the polypeptide can be generally selected from coagulation modulators, cytokines, endorphins, kinins, hormones, luteinizing hormone-releasing hormone analogs and others. Coagulation modulators include, for example, α-1-antitrypsin, α-2-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, thrombomodulin and combinations thereof. When applicable, both the “active” and “inactive” versions of these proteins are included.

Preferred cytokines include, without limitation, colony stimulating factor 4, heparin binding neurotrophic factor (HBNF), interferons, interleukins, tumor necrosis factor, granuloycte colony-stimulating factor (G-CSF), granulocyte-macrophage colony-stimulating factor (GM-CSF), macrophage colony-stimulating factor, midkine (MD), thymopoietin and combinations thereof.

Preferred endorphins include, but are not limited to, dermorphin, dynorphin, α-endorphin, β-endorphin, γ-endorphin, σ-endorphin, enkephalin, substance P, and combinations thereof.

Preferred peptidyl hormones include activin, amylin, angiotensin, atrial natriuretic peptide (ANP), calcitonin, calcitonin gene-related peptide, calcitonin N-terminal flanking peptide, cholecystokinin (CCK), ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, ghrelin, glucogon, gonadotropin-releasing factor (GnRF or GNRH), growth hormone releasing factor (GRF, GRH), human chorionic gonadotropin (hCH), inhibin A, inhibin B, insulin, leptin, lipotropin (LPH), luteinizing hormone (LH), luteinizing hormone-releasing hormone, melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, triiodothyronine, vasoactive intestinal peptide (VIP), vasopressin (antidiuretic hormone, ADH) and combinations thereof.

Particularly preferred analogues of LHRH include buserelin, deslorelin, fertirelin, goserelin, histrelin, leuprolide (leuprorelin), lutrelin, nafarelin, tryptorelin and combinations thereof. Particularly preferred kinins include bradykinin, potentiator B, bradykinin potentiator C, kallidin and combinations thereof.

Still other peptidyl drugs that provide a desired pharmacological activity can be incorporated into the delivery systems of the invention. Examples include abarelix, adenosine deaminase, anakinra, ancestim, alteplase, alglucerase, asparaginase, bivalirudin, bleomycin, bombesin, desmopressin acetate, des-Q14-ghrelin, dornase-α, enterostatin, erythropoietin, fibroblast growth factor-2, filgrastim, β-glucocerebrosidase, gonadorelin, hyaluronidase, insulinotropin, lepirudin, magainin I, magainin II, nerve growth factor, pentigetide, thrombopoietin, thymosin α-1, thymidin kinase (TK), tissue plasminogen activator, tryptophan hydroxylase, urokinase, urotensin II and combinations thereof.

Exendin-4 is a 39 amino acid polypeptide. The amino acid sequence of exendin-4 can be found in U.S. Pat. No. 5,424,286 issued to Eng on Jun. 13, 1995, the entire content of which is hereby incorporated by reference. Exendin-4 has been shown in humans and animals to stimulate secretion of insulin in the presence of elevated blood glucose concentrations, but not during periods of low blood glucose concentrations (hypoglycemia). It has also been shown to suppress glucagon secretion, slow gastric emptying and affect food intake and body weight, as well as other actions. As such, exendin-4 and analogs and agonists thereof can be useful in the treatment of diabetes mellitus, IGT, obesity, etc.

The amount of biologically active polypeptide, which is contained within the polymeric matrix of a sustained release composition, is a therapeutically, diagnostically or prophylactically effective amount which can be determined by a person of ordinary skill in the art, taking into consideration factors such as body weight, condition to be treated, type of polymer used, and release rate from the polymer.

Sustained release compositions generally contain from about 0.01% (w/w) to about 50% (w/w) of the agent, e.g., biologically active polypeptide (such as exendin-4) (total weight of composition). For example, the amount of biologically active polypeptide (such as exendin-4) can be from about 0.1% (w/w) to about 30% (w/w) of the total weight of the composition. The amount of polypeptide will vary depending upon the desired effect, potency of the agent, the planned release levels, and the time span over which the polypeptide will be released. Preferably, the range of loading is between about 0.1% (w/w) to about 10% (w/w), for example, 0.5% (w/w) to about 5% (w/w). Superior release profiles were obtained when the agent, e.g. exendin-4, was loaded at about 3 to 5% w/w.

The Polymer

Polymers suitable to form the sustained release composition of this invention are biocompatible polymers which can be either biodegradable or non-biodegradable polymers or blends or copolymers thereof. A polymer is biocompatible if the polymer and any degradation products of the polymer are non-toxic to the recipient and also possess no significant deleterious or untoward effects on the recipient's body, such as a substantial immunological reaction at the injection site.

Biodegradable, as defined herein, means the composition will degrade or erode in vivo to form smaller units or chemical species. Degradation can result, for example, by enzymatic, chemical and physical processes. Suitable biocompatible, biodegradable polymers include, for example, poly(lactides), poly(glycolides), poly(lactide-co-glycolides), poly(lactic acid)s, poly(glycolic acid)s, polycarbonates, polyesteramides, polyanydrides, poly(amino acids), polyorthoesters, poly(dioxanone)s, poly(alkylene alkylate)s, copolymers or polyethylene glycol and polyorthoester, biodegradable polyurethane, blends thereof, and copolymers thereof.

Suitable biocompatible, non-biodegradable polymers include non-biodegradable polymers selected from the group consisting of polyacrylates, polymers of ethylene-vinyl acetates and other acyl substituted cellulose acetates, non-degradable polyurethanes, polystyrenes, polyvinylchloride, polyvinyl flouride, poly(vinyl imidazole), chlorosulphonate polyolefins, polyethylene oxide, blends thereof, and copolymers thereof.

Acceptable molecular weights for polymers used in this invention can be determined by a person of ordinary skill in the art taking into consideration factors such as the desired polymer degradation rate, physical properties such as mechanical strength, end group chemistry and rate of dissolution of polymer in solvent. Typically, an acceptable range of molecular weight is of about 2,000 Daltons to about 2,000,000 Daltons. In a preferred embodiment, the polymer is biodegradable polymer or copolymer. In a more preferred embodiment, the polymer is a poly(lactide-co-glycolide) (hereinafter “PLG”) with a lactide:glycolide mole ratio of about 1:1 and a molecular weight of about 10,000 Daltons to about 90,000 Daltons. A PLG copolymer with a 1:1 lactide:glycolide mole ratio can also be referred to as a 50:50 PLG. In an even more preferred embodiment, the PLG used in the present invention has a molecular weight of about 30,000 Daltons to about 70,000 Daltons such as about 50,000 to about 60,000 Daltons.

The PLGs can possess acid end groups or blocked end groups, such as can be obtained by means known in the art, including esterifying the acid.

Polymers can also be selected based upon the polymer's inherent viscosity. Suitable inherent viscosities include about 0.06 to 1.0 dL/g, such as about 0.2 to 0.6 dL/g, more preferably between about 0.3 to 0.5 dL/g. Preferred polymers are chosen that will degrade in 3 to 4 weeks. Suitable polymers can be purchased from Lakeshore Biomaterials, Inc. (Birmingham, Ala.), such as those sold as 5050 DL 3A or 5050 DL 4A. BOEHRINGER INGELHEIM RESOMER® PLGs may also be used, such as RESOMER® RG503 and 503H.

It is known in the art (see, for example, Peptide Acylation by Poly(α-Hydroxy Esters) by Lucke et al., Pharmaceutical Research, Vol. 19, No. 2, p. 175-181, February 2002) that proteins and peptides which are incorporated in PLG matrices can be undesirably altered (e.g., degraded or chemically modified) as a result of interaction with degradation products of the PLG or impurities remaining after preparation of the polymer, such as, for example, unreacted lactide or glycolide. As such, the PLG polymers used in the preparation of microparticle formulations may be purified using art recognized purification methods.

The sustained release composition of this invention is a microparticle. A microparticle, as defined herein, comprises a polymer component having a diameter of less than about one millimeter and having biologically active polypeptide dispersed or dissolved therein. A microparticle can have a spherical, non-spherical or irregular shape. Typically, the microparticle will be of a size suitable for injection. A typical size range for microparticles is 1000 microns or less. In a particular embodiment, the microparticle ranges from about one to about 180 microns in diameter. Microparticles possessing a narrow size distribution are preferred.

Additional Excipients

Additional excipients or additives may be included in the microparticle compositions of the invention to serve a multiplicity of functions, including stabilization of the active agent during the encapsulation process, during storage prior to use, or during the period after injection and prior to release when the active agent resides in the microparticle at body temperature under moist conditions. Moreover, excipients can increase or decrease the rate of release of the agent. Ingredients which can substantially increase the rate of release include pore forming agents and excipients which facilitate polymer degradation. For example, the rate of polymer hydrolysis is increased in non-neutral pH. Therefore, an acidic or a basic excipient such as an inorganic acid or inorganic base can be added to the polymer solution, used to form the microparticles, to alter the polymer erosion rate. Ingredients which can substantially decrease the rate of release include excipients that decrease the water solubility of the agent. Excipients may also be employed to improve the biocompatibility and local tolerability of the microparticle composition.

Suitable excipients include, for example, salts, including buffer salts, sugars, carbohydrates, and surfactants, and are known to those skilled in the art. An acidic or a basic excipient may also be suitable. The amount of excipient used can be based on ratio to the biologically active polypeptide agent, on a weight basis and can be determined by one of skill in the art using available methods. Alternatively, the amount of excipient can be based on its content as a percent of the microparticle dry weight. The combined loading of the active agent and the excipient, or excipients if more than one is present, may have an effect on the release profile of the active agent. For example, when the combined loading of the active agent and the excipients exceeds about 10% of the total dry weight of the microparticles, a greater portion of drug may be released immediately upon suspension of the microparticles in a diluent for injection, or during the first day after injection. In a preferred embodiment, the combined loading of active agent and excipients is less than about 10% of the total dry weight of the microparticles. In a more preferred embodiment, the combined loading of active agent and excipients is from about 3% to about 8% of the total dry weight of the microparticles. In a still more preferred embodiment, the combined loading of active agent and excipients is from about 5% to about 7% of the total dry weight of the microparticles. Superior release profiles were obtained when an active agent, e.g. exendin-4, was loaded together with sucrose at a combined loading of about 5% w/w.

Excipients can be incorporated into the microparticle compositions of the present invention by several different means. In a preferred embodiment, water-soluble excipients are dissolved together with the active agent in water and then dispersed in the polymer solution prior to the addition of the coacervation agent. Alternatively, excipients can be added as solids at any stage of the process, or dissolved in the polymer solution, or dissolved in water and dispersed in the polymer solution separately from the active agent.

Salts

Buffer salt, as defined herein is the salt remaining following removal of solvent from a buffer. Buffers are solutions containing either a weak acid and a related salt of the acid, or a weak base and a salt of the base. Buffers can maintain a desired pH to assist in stabilizing the formulation. This maintenance of pH can be afforded during processing, storage and/or release. For example, the buffer can be monobasic phosphate salt or dibasic phosphate salt or combinations thereof or a volatile buffer such as ammonium bicarbonate. Other buffers include, but are not limited to, acetate, citrate, succinate and amino acids such as glycine, arginine and histidine. The buffer when present in the final sustained release composition can range from about 0.01% to about 10% of the total weight.

Salting-out salts can also be employed as excipients in the compositions of the present invention. Salting-out salts, as that term is used herein, refers to salts which are in the Hofmeister series of precipitants of serum as described in Thomas E. Creighton In Proteins: Structures and Molecular Principles, pp. 149-150 (published by W.H. Freeman and Company, New York). In general, the salting-out salts are known in the art as suitable for precipitating a protein, without denaturing the protein. Salting-out salts can also be described in terms of the “kosmotrope” and “chaotrope” properties of the constituent ions. The term kosmotrope generally refers to a solute that stabilizes proteins and chaotrope describes a solute that is destabilizing. Kosmotropic ions have a high charge density (e.g., SO₄²⁻, HPO₄²⁻, Mg²⁺, Ca²⁺, Li⁺, Na⁺ and HPO₄²⁻) and chaotropic ions have a low charge density (examples include H₂PO₄⁻, HSO₄⁻, HCO₃⁻, I⁻, Cl⁻, NO₃⁻, NH₄⁺, Cs⁺, K⁺, [N(CH₃)₄]⁺). The salting out salt can also be described in terms of its ability to donate or accept protons, and as such acting as a base or acid. For instance, the salting out salt (NH₄)₂SO₄ provides an ammonium ion, and can act as an inorganic acid. When included in a polymeric microparticle such inorganic acids can modulate polymer degradation and affect release of incorporated agent. In certain embodiments, amino acids such as glycine which is considered in the art as a kosmotrope can be used as an alternative to the salting-out salt.

Suitable salting-out salts for use in this invention include, for example, salts containing one or more of the cations Mg⁺², Li⁺, Na⁺, K⁺ and NH₄⁺; and also containing one or more of the anions SO₄⁻², HPO₄⁻², acetate, citrate, tartrate, Cl⁻, NO₃⁻, ClO₃⁻, I⁻, ClO₄⁻ and SCN⁻.

The amount of salting-out salt present in the sustained release composition can range from about 0.01% (w/w) to about 50% (w/w), such as from about 0.01% to about 10% (w/w), for example from about 0.01% to about 5%, such as 0.1% to about 5% of the total weight of the sustained release composition. Combinations of two or more salting-out salts can be used. The amount of salting-out salt, when a combination is employed, is the same as the range recited above.

Sugars

A sugar, as defined herein, is a monosaccharide, disaccharide or oligosaccharide (from about 3 to about 10 monosaccharides) or a derivative thereof. For example, sugar alcohols of monosaccharides are suitable derivatives included in the present definition of sugar. As such, the sugar alcohol mannitol, for example, which is derived from the monosaccharide mannose is included in the definition of sugar as used herein.

Suitable monosaccharides include, but are not limited to, glucose, fructose and mannose. A disaccharide, as further defined herein, is a compound which upon hydrolysis yields two molecules of a monosaccharide. Suitable disaccharides include, but are not limited to, sucrose, lactose and trehalose. Suitable oligosaccharides include, but are not limited to, raffinose and acarbose.

The amount of sugar present in the sustained release composition can range from about 0.01% (w/w) to about 50% (w/w), such as from about 0.01% (w/w) to about 10% (w/w), such as from about 0.1% (w/w) to about 5% (w/w) of the total weight of the sustained release composition. Excellent release profiles were obtained incorporating about 2% (w/w) sucrose in a microparticle loaded with exendin-4.

Alternatively, the amount of sugar present in the sustained release composition can be referred to on a weight ratio with the agent or biologically active polypeptide. For example, the polypeptide and sugar can be present in a ratio from about 10:1 to about 1:10 weight:weight. In a particularly preferred embodiment, the ratio of polypeptide (e.g., exendin-4) to sugar (e.g., sucrose) is about 5:2 (w/w).

Combinations of two or more sugars can also be used. The amount of sugar, when a combination is employed, is the same as the ranges recited above.

When the polypeptide is exendin-4, the sugar is preferably sucrose, mannitol or a combination thereof.

Surfactants

A surfactant can be present in the sustained release composition. The surfactant can act to further modify release of the biologically active polypeptide from the polymer matrix, or can act to further stabilize the biologically active polypeptide or a combination thereof. The presence of surfactant can in some instances assist in minimizing adsorption of the biologically active polypeptide to the biocompatible polymer. The amount of surfactant present in the sustained release composition can range from about 0.1% w/w to about 50% w/w of the dry weight of the composition.

Surfactants, as the term is used herein, include substances which can reduce the surface tension between immiscible liquids. Suitable surfactants which can be added to the sustained release composition include polymer surfactants, such as nonionic polymer surfactants, for example, poloxamers, polysorbates, polyethylene glycols (PEGs), polyoxyethylene fatty acid esters, polyvinylpyrrolidone and combinations thereof. Examples of poloxamers suitable for use in the invention include poloxamer 407 sold under the trademark PLURONIC® F127, and poloxamer 188 sold under the trademark PLURONIC® F68, both available from BASF Wyandotte. Examples of polysorbates suitable for use in the invention include polysorbate 20 sold under the trademark TWEEN® 20 and polysorbate 80 sold under the trademark TWEEN® 80.

Cationic surfactants, for example, benzalkonium chloride, are also suitable for use in the invention. In addition, bile salts, such as deoxycholate and glycocholate are suitable as surfactants based on their highly effective nature as detergents.

Exendin-4 Compositions

A preferred embodiment of the present invention is a composition for sustained delivery of exendin-4 made by the processes disclosed herein, and comprising a biocompatible polymer, the active agent, and a sugar.

Manufacturing Processes

The present invention relates to methods of forming compositions for the sustained delivery of active agents and the products produced thereby. These methods are based on a coacervation process, which includes forming a first phase comprising the active agent, the polymer and a solvent; adding a coacervation agent, for example silicone oil, vegetable oil or mineral oil to the first phase to form embryonic microparticles; transferring the embryonic microparticles to a quench solvent to harden the microparticles; collecting the hardened microparticles; and drying the hardened microparticles. The first phase can be a water-in-oil emulsion prepared by combining an aqueous solution of the active agent with a solution of the polymer in an organic solvent. In this case, the process is generally referred to herein as a water-oil-oil process (W/O/O). Alternatively, the first phase can be a suspension of solid particles of the active agent in a solution of the polymer in an organic solvent. This alternative process is referred to herein as a solid-oil-oil process (S/O/O).

Preferably, the polymer can be present in the organic solvent at a concentration ranging from about 3% w/w to about 25% w/w, preferably, from about 4% w/w to about 15% w/w, such as from about 5% w/w to about 10% w/w. Where the polymer is a PLG, such as those preferred herein, the polymer is dissolved in a solvent for PLG. Such solvents are well known in the art, and are selected from the group consisting of alcohols, esters, ketones, halogenated hydrocarbons and blends thereof. Preferred solvents are methylene chloride and ethyl acetate.

The agent and water soluble excipients, such as a sugar, are typically added in the aqueous phase, preferably in the same aqueous phase. The concentration of agent is preferably 10 to 100 mg/g, more preferably between 50 to 100 mg/g. The concentration of sugar is preferably 10 to 50 mg/g and more preferably 30 to 50 mg/g.

The solutions of the active agent and polymer are then mixed to form a water-in-oil emulsion, which is referred to herein as a first phase. It is preferred that the first phase emulsion be formed such that the inner emulsion droplet size is less than about 1 micron, preferably less than about 0.7 microns, more preferably less than about 0.5 microns, such as about 0.4 microns. Sonicators and homogenizers can be used to form such an emulsion.

In the embodiment of the coacervation process wherein the active agent is dispersed in the polymer solution as a solid (i.e., the S/O/O process), it is preferred that the suspended drug particle diameter be less than about 10 microns, preferably less than about 5 microns, and more preferably less than about 1 micron. Methods of producing submicron particles of biologically active agents are known in the art. For example, U.S. Pat. No. 6,428,815 discloses a spray-freeze drying process capable of producing friable microstructures which can be fragmented in the polymer solution to achieve submicron particles by means known to those skilled in the art, for example by probe sonication, homogenization, fluidization, comminution and milling.

Coacervate Formation

A coacervation agent as used herein refers to a non-solvent for the polymer that is miscible with the polymer solvent. Coacervation agents may be low molecular weight polymer non-solvents. Alternatively, the coacervation agent may be a second polymer that is incompatible with the polymer that forms the microparticle. Addition of a coacervation agent reduces the solubility of the polymer, causing it to undergo phase separation, thus forming a coacervate. Suitable coacervation agents for use in the present invention include, but are not limited to, silicone oil, vegetable oil and mineral oil. In a particular embodiment, the coacervation agent is silicone oil and the polymer solvent is methylene chloride. Silicone oil is added in an amount sufficient to achieve a final silicone oil to methylene chloride ratio from about 0.75:1 to about 2:1. In a preferred embodiment, the final ratio of silicone oil to methylene chloride is from about 1:1 to about 1.5:1. In a more preferred embodiment, the final ratio of silicone oil to methylene chloride is about 1.3:1. A preferred silicone oil is dimethicone having a viscosity of between about 100 and 1000 centistokes, preferably about 350 centistokes. In processes that employ coacervation agents other than silicone oil or polymer solvents other than methylene chloride, a suitable ratio of coacervation agent to polymer solvent can be selected on the basis of experimentally determined effects of the amount of coacervation agent on the microparticle size distribution, residual solvent and coacervation agent levels, and drug release kinetics.

The behavior of the nascent microparticles during formation of the coacervate is dependent on the process conditions. At low coacervation agent to polymer solvent ratios, e.g., after partial addition of the coacervation agent, the particle size of the nascent microparticles tends to be relatively stable. However, at higher ratios of coacervation agent to polymer solvent, e.g., near or prior to the completion of coacervation agent addition, nascent microparticles are prone to growth or aggregation, which can result in an unacceptably broad particle size distribution in the final microparticle composition, or unacceptable yield losses if a finishing step is performed to remove oversized particles. With specific regard to the case where the coacervation agent is silicone oil, the polymer is PLG and the solvent is methylene chloride, at silicone oil to methylene chloride ratios of 0.5:1 or less, the particle size of the nascent microparticles is comparatively stable. At silicone oil to methylene chloride ratios of 0.7:1 or greater, the particle diameter increases over time. It is therefore preferred to limit the time interval during the coacervation step wherein the ratio of coacervation agent to polymer solvent is high enough to promote growth of the nascent particles.

When the polymer solvent is methylene chloride and the coacervation agent is silicone oil, this time interval is preferably less than 10 minutes, and more preferably less than 5 minutes. Accordingly, an aspect of the present invention is coacervation processes wherein the time interval during which the coacervation agent to polymer solvent ratio is high enough to allow growth of the nascent particles is kept sufficiently short.

The rate of addition of coacervation agent to the first phase, and the efficiency with which the coacervation agent and the first phase are blended, can also impact characteristics for the microparticle composition. In the case where the coacervation agent is silicone oil, the polymer is PLG and the solvent is methylene chloride, high mass flow rates of silicone oil and/or slow silicone oil blend times can result in high residual silicone oil levels in the microparticles due to entrapment of silicone oil in the nascent microparticles. The level of residual silicone oil in the microparticles is dependent on the addition rate at the early stages of silicone oil addition, for example when the ratio of silicone oil to methylene chloride is 0.5:1 or less. In order to control the level of residual silicone oil in the microparticles, silicone oil is preferably added slowly up to a ratio of silicone oil to methylene chloride of, for example, about 0.375:1. The rate of silicone oil addition is selected such that this phase of silicone oil addition occurs over a time interval of preferably greater than about 3 minutes, and more preferably over a period of at least about 5 minutes. In processes wherein the polymer solvent is not methylene chloride or the coacervation agent is not silicone oil, the minimum time for coacervation agent addition can be determined experimentally using methods known in the art. The early stages of coacervation agent addition to the first phase are preferably conducted under conditions where the coacervation agent is well dispersed in and efficiently blended with the first phase. Means of increasing the efficiency of blending include conducting the coacervation step in a stirred tank reactor outfitted with multiple addition ports or by adding the coacervation agent to the first phase through one or more spray nozzles.

Accordingly, an aspect of the present invention is a coacervation process wherein the addition of a coacervation agent to the first phase is conducted in at least two stages. The early stage or stages of coacervation agent addition takes place under conditions that avoid entrapment of coacervation agent in the microparticles, e.g., by slow addition of coacervation agent using equipment that promotes efficient blending of the coacervation agent with the first phase. Nascent microparticles characterized by a comparatively stable microparticle size are formed during the first stage.

The later stage or stages of the coacervation step take place under conditions designed to control particle size growth, such as minimizing the time interval during which the ratio of coacervation agent to polymer solvent may be high enough to promote particle size growth. For example, in the case of silicone oil, PLG and methylene chloride, the addition time during the second stage is less than 10 minutes, preferably less than 5 minutes. The silicone oil is preferably added to achieve a final oil:solvent ratio of at least 1:1, preferably at least 1.3:1, thereby ensuring low burst and residual impurities.

The individual stages of coacervation agent may all take place in a single apparatus, or they may take place as separate unit operations in distinct pieces of equipment. Moreover, the individual stages of coacervation agent addition may all be conducted in either batch mode or continuous mode, or certain stages may be run as batch operations and others as continuous processes. Furthermore, it may be appropriate to introduce hold times between different stages of coacervation agent addition in order to allow time dependent processes to occur, such as, for example, diffusion of the polymer solvent out of the nascent microparticle. Alternatively different stages of coacervation agent addition may be made to take place in immediate succession, with no hold time in between. In this embodiment, however, it will be clear that the rates of silicone oil addition will differ between the two stages. Preferably, the rate of addition during the first stage will be less than that of the second stage.

In addition, a further aspect of the present invention is processes wherein the same coacervation agent is used for all stages of coacervation agent addition. An alternative aspect of the invention is when different coacervation agents are employed at different stages of coacervation agent addition. Coacervation agents used at different stages of addition could differ with respect to the type of oil, for example vegetable oil versus silicone oil, the viscosity of the oil, for example 350 cSt silicone oil versus 1000 cSt silicone oil, or the presence or absence of additives to the coacervation agent. Such additives may include surfactants, including but not limited to lipophilic surfactants such as the series of fatty acyl sorbitans known as SPAN® detergents.

Batch Mode Multistage Coacervation Process

An embodiment of the multistage coacervation process of the invention is the process wherein the addition of the coacervation agent takes place in a single stirred tank reactor in a batch mode. In this case, the reactor should be sized appropriately to accommodate the entire amount of first phase and coacervation agent required to produce a batch. The first phase is either prepared directly in the stirred tank, or preferably, introduced into the stirred tank from an apparatus designed to produce a submicron dispersion of drug particles or drug solution droplets in the polymer solution, using, for example homogenization or sonication.

Coacervation agent is added in two or more sequential stages. The initial stage or stages of coacervation agent addition are preferably performed at a slow addition rate and with efficient dispersion in order to preclude entrapment of coacervation agent in the nascent microparticles. In a preferred embodiment, the coacervation agent is added through multiple addition ports or through one or more spray nozzles. Addition ports should be sized and arranged appropriately to promote efficient blending of coacervation agent with the first phase. Suitable spray nozzles include single-fluid nozzles, which are operated at differential pressures sufficient to disperse the coacervation agent in the first phase (10-60 psi). The amount of coacervation agent added is limited in order to maintain a coacervation agent to polymer solvent ratio that is low enough to preclude or minimize particle size growth. The resultant mixture of first phase and coacervation agent is referred to herein as an intermediate coacervate dispersion. During the last stage or stages of coacervation agent addition, coacervation agent is added to the intermediate coacervate dispersion until the final ratio of coacervation agent to polymer solvent is achieved. The addition rate of coacervation agent and the time to transfer the coacervate into the quench liquid for hardening are preferably selected such that the time interval during which the ratio of coacervation agent to polymer solvent is above the maximum ratio for stable particle size is short; preferably less than 5 minutes.

The time interval between successive stages of coacervation agent addition may be optimized with respect to product yield or selected on the basis of other final product attributes, including particle size, residual solvent levels or active agent release kinetics. This time interval may be essentially zero, meaning that two stages of coacervation agent addition occur in immediate succession. The delineation between stages may be a step change in the rate of coacervation agent addition, or a change in addition rate effected by a ramp in pump speed over a period of time. Alternatively, a hold time during which no coacervation agent is added may take place between stages, in order to allow time-dependent processes to occur, such as, for example, diffusion of the polymer solvent out of the nascent microparticle.

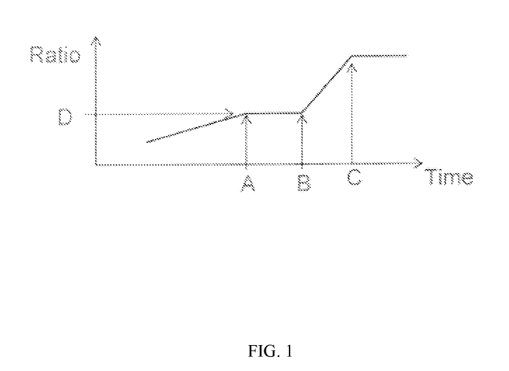

This embodiment includes a process wherein the addition of the coacervation agent to the first phase to form the coacervate comprises exactly two sequential stages of coacervation agent addition. The first stage is preferably conducted over a period of at least about 3 minutes, and more preferably at least about 5 minutes. The amount of coacervation agent added during this stage is approximately 75-100% of the maximum amount that can be added while maintaining a stable particle size of the nascent microparticles. In the embodiment wherein the coacervation agent is silicone oil and the polymer solvent is methylene chloride, this ratio is from about 0.375:1 to about 0.5:1. The second and final stage of coacervation agent addition is conducted over a period of less than 5 minutes and the final ratio of silicone oil to methylene chloride is from about 0.75:1 to about 2:1. In a preferred embodiment, the final ratio of silicone oil to methylene chloride is from about 1:1 to about 1.5:1. In a more preferred embodiment, the final ratio of silicone oil to methylene chloride is between about 1.3:1 and 1.7:1, such as about 1.5:1. The time course of coacervation agent addition in this process is depicted in FIG. 1, in which the ratio of coacervation agent to polymer solvent is plotted against time. Time A is greater than about 3 minutes; ratio D is 0.375:1, and the time interval from time B to C is less than about 5 minutes.

Continuous Multistage Coacervation Process

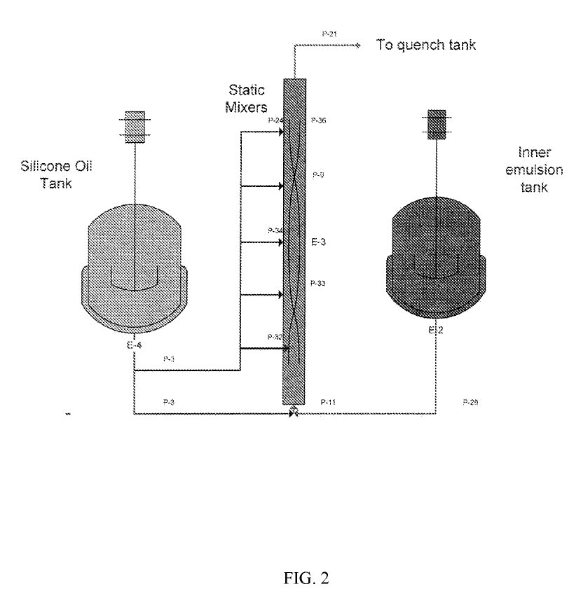

An alternative embodiment of the present invention is a continuous coacervation process wherein coacervation agent is added to the first phase in at least two stages in a continuous flow multistage mixing apparatus. One example is multiple static mixers deployed in series, as illustrated in FIG. 2. The use of static mixers in microencapsulation processes is disclosed in U.S. Pat. No. 5,654,008, which is incorporated herein by reference. The first phase is combined with a first coacervation agent and fed into a static mixer. The outflow is combined with a second coacervation agent at the entrance to a second static mixer. For purposes of illustration, FIG. 2 depicts a process wherein a single coacervation agent is combined with the first phase at the inlet of the static mixer assembly and at 5 additional inlets. The number of static mixers, the geometry of each mixer and the flow rates of first phase and each coacervation agent stream can be optimized to provide the desired particle size and production rates. Moreover, the same coacervation agent can be introduced at different inlets, as depicted in the figure, or different coacervation agents may be employed. Similarly, the flow rate of coacervation agent may be the same at all of the inlets, or it may be different. Variations of this concept include a single static mixer with multiple coacervation agent addition ports along its length, and a single static mixer with porous walls through which the coacervation agent is added continuously along the length of the static mixer. The porous wall addition could be through the outside wall of the static mixer or through a porous center tube in the middle of the static mixer.

A single static mixer or series of static mixers may be employed, or multiple static mixers may be deployed in parallel. The ability to use multiple static mixers in parallel simplifies scale-up, since the flow parameters through each individual static mixer can be kept constant while the number of static mixers is increased in direct proportion to the batch size.

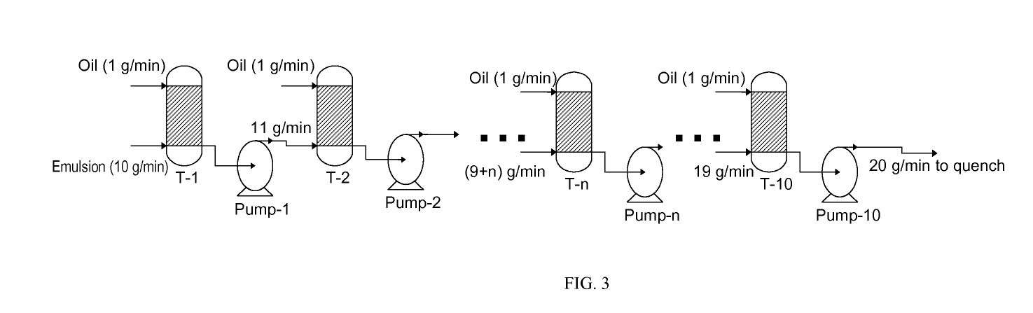

Another example is a continuous multistage process employing multiple CSTRs in series, as shown in FIG. 3. Here the first phase and a coacervation agent are fed into the first CSTR (T-1) and the outflow of this reactor is pumped (Pump-1) into a second CSTR (T-2), which also has a continuous coacervation agent addition. The intermediate coacervate from the second reactor is combined with additional coacervation agent in a third CSTR, and so forth. The series depicted in FIG. 3 for illustrative purposes consists of 10 CSTRs (of which 4 are shown in the diagram). The coacervate, which flows out of the last reactor (T-10 in FIG. 3) is subsequently contacted with a quench liquid to harden the microparticles. The composition and flow rate of each coacervation agent may be the same or may differ. The number and size of the reactors, the flow rates and agitation conditions determines the time course of addition of coacervation agent or agents during formation of the nascent microparticles.

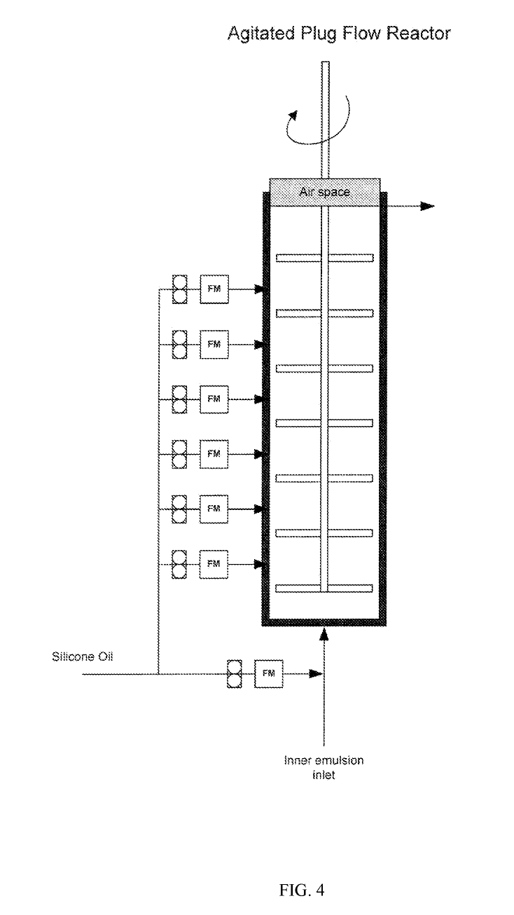

An agitated plug flow reactor consisting of a single, tall mixing vessel with multiple impellors and multiple coacervation agent injection ports is depicted in FIG. 4. The first phase is combined with a coacervation agent allowed to flow into the reactor. Additional coacervation agent is introduced through each injection port. Each impellor and coacervation agent addition zone acts as a mixing compartment within the vessel. The number and size of the mixing compartments, the flow rates of first phase and coacervation agents and the agitation parameters (impellor geometry and speed) determine the particle size and production rates.

Additional continuous multistage coacervation processes comprising aspects of any or all of these three configurations are readily apparent to one of skill in the art. In all cases, the profile of coacervation agent addition can be modified to optimize product properties. In all cases, the mixing shear rate can be adjusted to optimize particle size and size distribution. For static mixers, this can be achieved by varying the flow rates, the diameter of the static mixer, or the design of the static mixer. For the agitated options this can be controlled by the mixer speed and the choice of agitator blades. Excessive shear can cause nascent microparticles to lose their structure and form strands and should therefore be avoided.

Hybrid Multistage Coacervation Process

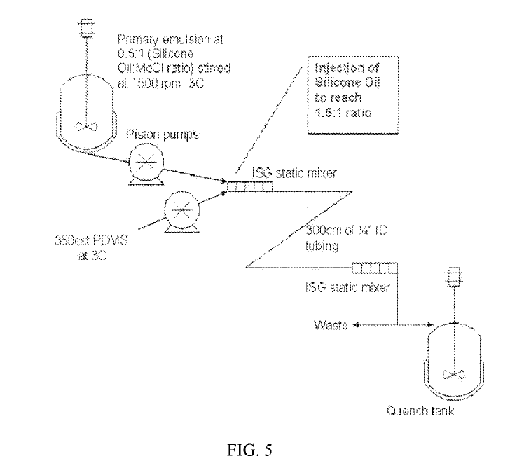

A preferred embodiment of the present invention is a hybrid process wherein the first stage or stages of coacervation agent addition are conducted in a stirred tank reactor, and the final stage or stages are conducted in a continuous flow mixing apparatus; preferably a static mixer. The coacervate is then combined with a quench liquid comprising a hardening agent. An experimental setup illustrative of this embodiment process is in FIG. 5. Here the first phase containing the active agent is combined with a coacervation agent in a stirred tank reactor, preferably under conditions described previously to minimize entrapment of coacervation agent, including slow addition of coacervation agent and efficient blending of the coacervation agent with the first phase. The amount of coacervation agent added during the first stage or stages of addition is preferably about 75-100% of the maximum amount that can be added while maintaining a stable particle size of the nascent microparticles.

The resulting intermediate coacervate is pumped out of the stirred tank and combined with an additional coacervation agent stream and made to flow through a static mixer or assembly comprising multiple static mixers or a combination of static mixers and empty tubing. FIG. 5 depicts a process wherein the mixture of coacervation agent and the intermediate coacervate are mixed in an assembly consisting of 2 static mixers bracketing a length of hollow tubing.

The static mixer assembly is designed in order (1) to provide sufficient throughput to enable processing of all of the intermediate coacervate within the time interval during which the particle size of the nascent microparticles is stable, (2) to provide sufficient mixing of the coacervating agent with the intermediate coacervate, and (3) to provide sufficient residence time to allow extraction of the polymer solvent from the nascent microparticles.

In the process illustrated in FIG. 5, the outflow of the static mixer assembly is directed into a quench tank containing the hardening agent. Alternatively, the coacervate outflow can be mixed with a quench liquid upstream of an additional static mixer to enable continuous hardening of the microparticles as discussed below.

The hybrid process allows the addition rate of the first coacervation agent to be as slow as necessary in order to avoid entrapment of coacervation agent and resulting high residual coacervation agent levels in the microparticles. Subsequent addition of coacervation agent is conducted in a continuous manner, so that the time interval during which the coacervate dispersion can be maintained at the final ratio of coacervation agent to polymer solvent is equal to the residence time of the continuous flow mixing apparatus. This minimizes the particle size growth observed at high ratios of coacervation agent to polymer solvent.

Microparticle Hardening

The coacervate formed either by a multistage process of this invention or by a conventional single stage process, is combined with a quench liquid and the nascent microparticles are allowed to harden. The quench liquid typically comprises a hardening agent, which is a non-solvent for the polymer but is miscible with the coacervation agent and the polymer solvent. Polymer non-solvents are generally well known in the art. Preferred hardening agents include liquid hydrocarbons. Of these, heptane is particularly preferred. Other components of quench liquids known in the art include alcohols, including ethanol. A particularly preferred quench comprises a heptane/ethanol solvent system, for example as described in U.S. Pat. No. 6,824,822, which is incorporated herein by reference.

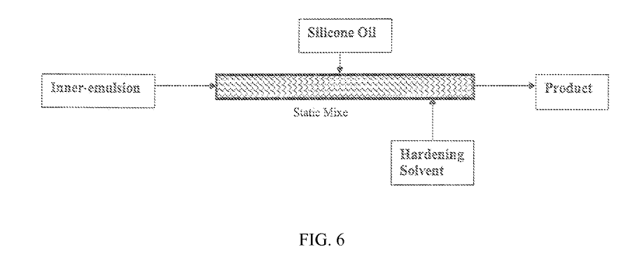

An aspect of the current invention is the process wherein the coacervate is combined with a quench liquid in a continuous process using a static mixer. An example of this process is shown in FIG. 6. A first phase is combined with a coacervation agent at the entrance of a static mixer. At a point downstream, the quench liquid is introduced into the static mixer and is mixed with the coacervate. The mixture of coacervate and quench liquid is passed through an additional length of static mixer and then directed into a quench tank for additional hardening or washing, or into a collection device such as a filter drier.

Variations of this process are readily apparent to one of skill in the art. In one such embodiment, a first static mixer is employed to combine the first phase with a coacervation agent; the resulting coacervate is combined with a quench liquid at the entrance to a second static mixer; and the outflow is directed into a quench tank. In a preferred embodiment, the coacervate formed in the hybrid multistage process of this invention is mixed in a continuous manner with the quench liquid in an additional static mixer and then directed into a quench tank.

Use of a static mixer to combine the coacervate with the hardening agent improves the scaleability of the process and affords a potential reduction in the consumption of hardening liquid.

The invention will now be further and specifically described by the following examples.

### EXAMPLES

### Example 1

The effect of the profile of coacervation agent addition on residual silicone oil levels was assessed by producing placebo microparticle batches at the 105 gram and 1 kg and 15 kg scales. In addition, a 15 kg batch was produced using a spray nozzle and/or multiple addition ports to add the silicone oil to the first phase.

### 105 Gram Batch Size

### A. Inner Water-In-Oil Emulsion Formation

A water-in-oil emulsion was created with the aid of a sonicator (Vibracell VCX 750 with a ½″ probe (part #A07109PRB; Sonics and Materials Inc., Newtown, Conn.). The water phase of the emulsion was prepared by dissolving 2.1 g sucrose in 63 g water. The oil phase of the emulsion was prepared by dissolving PLG polymer (97.7 g of purified 50:50 DL4A PLG having an internal viscosity of about 0.45 dL/g (Alkermes, Inc. in methylene chloride (1530 g or 6% w/v).

The water phase was then added to the oil phase over about a three minute period while sonicating at 100% amplitude at ambient temperature. The reactor was then stirred at 1400 to 1600 rpm, with additional sonication at 100% amplitude for 2 minutes, followed by a 30 second hold, and then 1 minute more of sonication. This results in an inner emulsion droplet size of less than 0.5 microns.

### B. Coacervate Formation

A coacervation step was then performed by adding silicone oil (2294 g of Dimethicone, NF, 350 cs). This is equivalent to a ratio of 1.5:1, silicone oil to methylene chloride. The silicone oil was added in either a single step or in two steps separated by a hold time. The rate of silicone oil addition was varied as indicated in Table 1. The methylene chloride from the polymer solution partitions into the silicone oil and begins to precipitate the polymer around the water phase, leading to microencapsulation. The embryonic microspheres were permitted to stand for a short period of time, for example, from about 1 minute to about 5 minutes prior to proceeding to the microsphere hardening step.

### C. Microsphere Hardening and Rinse

The embryonic microspheres were then transferred into a mixture of about 22 kg heptane and 2448 g ethanol in a 3° C. cooled, stirred tank (350 to 450 rpm). This solvent mixture hardened the microspheres by extracting additional methylene chloride from the microspheres. After being quenched for 1 hour at 3° C., the solvent mixture was decanted and fresh heptane (13 kg) was added at 3° C. and held for 1 hour to rinse off residual silicone oil, ethanol and methylene chloride on the microsphere surface.

### D. Microsphere Drying and Collection

At the end of the rinse step, the microspheres were transferred and collected by filtration on a 20 or 25 micron screen. A final rinse with heptane (6 kg at 4° C.) was performed to ensure maximum line transfer. The microspheres were then vacuum dried with a purge of nitrogen gas. The temperature was increased according to the following schedule: 18 hours at 3° C.; 24 hours at 25° C.; 6 hours at 41° C.; and 42 hours at 45° C.

After the completion of drying, the microspheres were stored at −20±5° C. The yield was approximately 80 grams of microspheres.

### 1 kg Batch Size

### A. Inner Water-In-Oil Emulsion Formation

A water-in-oil emulsion was created with the aid of an in-line Megatron homogenizer MT-V 3-65 F/FF/FF, Kinematica AG, Switzerland. The water phase of the emulsion was prepared by dissolving 20 g sucrose in 600 g water for injection (WFI). The oil phase of the emulsion was prepared by dissolving PLG polymer (e.g., 930 g of purified 50:50 □L4A PLG (Alkermes, Inc.)) in methylene chloride (14.6 kg or 6% w/w).

The water phase was then added to the oil phase in a jacketed vessel cooled to 3° C. and mixed to form a coarse emulsion with an overhead mixer for about three minutes. Then the coarse emulsion was homogenized at approximately 10,000 rpm. This results in an inner emulsion droplet size of less than 1 micron.

### B. Coacervate Formation

A coacervation step was then performed by adding silicone oil (21.9 kg of Dimethicone, NF, 350 cs) to the inner emulsion. This is equivalent to a ratio of 1.5:1, silicone oil to methylene chloride. The rate of silicone oil addition was varied to test the effect of coacervation agent addition rate on residual silicone oil in the microparticles. Silicone oil addition rates for individual batches are listed in Table 1. The embryonic microspheres were permitted to stand for a short period of time, for example, from about 1 minute to about 5 minutes prior to proceeding to the microsphere hardening step.

### C. Microsphere Hardening and Rinse

The embryonic microspheres were then transferred into a heptane/ethanol solvent mixture. In the present example, a mixture of about 210 kg heptane and 23 kg ethanol in a 3° C. cooled, stirred tank was used. After quenching for 1 hour at 3° C., the solvent mixture was decanted and fresh heptane (55 kg) was added at 3° C. and held for 1 hour to rinse off residual silicone oil, ethanol and methylene chloride.

### D. Microsphere Drying and Collection

At the end of the quench or decant/wash step, the microspheres were transferred and collected on a 12″ Sweco Pharmasep Filter/Dryer Model PH12Y6. The filter/dryer uses a 20 micron multilayered collection screen and is connected to a motor that vibrates the screen during collection and drying. A final rinse with heptane (40 kg delivered in 4 10 kg aliquots at 3° C.) was performed to ensure maximum line transfer and to remove any excess silicone oil. The microspheres were then dried under vacuum with a constant purge of nitrogen gas at a controlled rate and the temperature increased according to the following schedule: 6 hours at 3° C.; 6 hours ramping to 41° C.; and 84 hours at 41° C.

After the completion of drying, the microspheres were discharged into a collection vessel, sieved through a 150 μm sieve, and stored at about −20° C. until filling.

### 15 kg Batch Size

### A. Inner Water-In-Oil Emulsion Formation

A water-in-oil emulsion was created with the aid of an in-line Megatron homogenizer MT-V 3-65 F/FF/FF, Kinematica AG, Switzerland. The water phase of the emulsion was prepared by dissolving 300 g sucrose in 9 kg water for irrigation (WFI). The oil phase of the emulsion was prepared by dissolving PLG polymer (e.g., 13,950 g of 50:50 □L4A PLG (Alkermes, Inc.) as described above) in methylene chloride (219 kg or 6% w/w).

The water phase was then added to the oil phase to form a coarse emulsion with an overhead mixer for about three minutes. Then, the coarse emulsion was homogenized at approximately 10,000 rpm at 5 C.

### B. Coacervate Formation

A coacervation step was then performed by adding silicone oil (330 kg of Dimethicone, NF, 350 cs) to the inner emulsion. This is equivalent to a ratio of 1.5:1, silicone oil to methylene chloride. Silicone oil addition parameters are listed in Table 1. The silicone oil was added in either a single step or in two steps separated by a hold time. In batches SAFC 066K7276 and SAFC 200-66-21A, silicone oil was added via a SpiralJet® spray nozzle (Spraying Systems Company, model HHSJ). Batch SAFC 200-66-27A was prepared using two addition ports for the first stage of silicone oil addition. The embryonic microspheres were permitted to stand for a short period of time, for example, from about 1 minute to about 5 minutes prior to proceeding to the microsphere hardening step.

### C. Microsphere Hardening and Rinse

The embryonic microspheres were then transferred into a heptane/ethanol solvent mixture. In the present example, about 3150 kg heptane and 350 kg ethanol in a 3° C. cooled, stirred tank were used. After being quenched for 1 hour at 3° C., the solvent mixture was decanted and fresh heptane (825 kg) was added at 3° C. and held for 1 hour to rinse off residual silicone oil, ethanol and methylene chloride.

### D. Microsphere Drying and Collection

At the end of the quench or decant/wash step, the microspheres were transferred and collected on a jacketed 0.2 m² filter dryer (3V Cogeim) equipped with a 20 micron Teflon membrane and a glycol-filled agitator for mixing and heat transfer during collection and drying. A final rinse with heptane (four 150 kg rinses at 3° C.) was performed to ensure maximum line transfer and to remove any excess silicone oil. The microspheres were then dried under vacuum with a constant purge of nitrogen gas at a controlled rate according to the following schedule: 6 hours at 3° C.; 36 hours ramping to 39° C.; and 30 hours at 39° C.

### Measurement of Residual Silicon

Residual silicon was measured by inductively coupled plasma spectroscopy (ICP; Galbraith Laboratories, Inc; Knoxyille, Tenn.). Results are listed in Table 1.

TABLE 1
Residual silicon oil levels in placebo microsphere batches

| | | | SiOil addition | | Residual |
|---|---|---|---|---|---|
| Lot # | Scale | | rate (kg/min) | Addition time (mm:ss) | silicon (ppm) |
| 200-00042-174 | 105 | g | 8.6 | 0:16 | 15000 |
| 200-00042-184A | 105 | g | 0.2; 8.3 | 3:49; 2 min hold; 0:11 | 384 |
| 200-00042-184B | 105 | g | 5.7; 9.2 | 0:08; 2 min hold; 0:10 | 2600 |
| 05-003-167 | 1 | kg | 5.7 | 3:50 | 78 |
| 05-016-027 | 1 | kg | 4.1 | 5:30 | 158 |
| 06-002-037 | 1 | kg | 0.9; 4.1 | 6:00; 5 min hold; 4:00 | 17 |
| SAFC 046K7278 | 15 | kg | 65 | 5:00 | 1600 |
| SAFC 056K7278 | 15 | kg | 110 | 3:00 | 2900 |
| SAFC 066K7276 | 15 | kg | 65 (w/nozzle) | 5:00 | 695 |
| SAFC 200-66-27A | 15 | kg | 28; 84 | 3:00; no hold; 3:00 | 428 |
| | | | (2 add'n ports) | | |
| SAFC 200-66-21A | 15 | kg | 14; 60 | 6:00; 5 min hold; 4:00 | 108 |
| | | | (w/nozzle) | | |

The particle size distribution obtained using a Coulter Multisizer of batch SAFC 200-66-21A after suspension with sonication in an aqueous diluent containing 3% carboxymethylcellulose, 0.9% NaCl, 0.1% Tween 20 was as follows: DV50—85.1 μm, DV90—120 μm, DV90-DV10—67.5 μm.

### Example 2

Microparticle batches containing exendin-4 were produced at 1 kg and 15 kg scales using a batch mode multistage coacervation process.

### 1 kg Batch Size

A water-in-oil emulsion was created in accordance with the 1 kg process described in Example 1 except that the water phase of the emulsion was prepared by dissolving 20 g sucrose and 50 grams exendin-4 in 600 g water for injection (WFI). Coacervation and subsequent processing steps were performed as described in Example 1. Coacervation agent was added in two distinct stages separated by a hold time as indicated in Table 2. For comparison, a reference batch was produced by adding the entire quantity of coacervation agent in a single stage. Residual silicone oil levels were determined and are listed in Table 2.

### 15 kg Batch Size

A water-in-oil emulsion was created in accordance with the 15 kg process described in Example 1 except that the water phase of the emulsion was prepared by dissolving 300 g sucrose and 750 grams exendin-4 in 600 g water for injection (WFI). Coacervation and subsequent processing steps were performed as described in Example 1. Coacervation agent was added via a spray nozzle as described in Example 1 in two distinct stages separated by a hold time as indicated in Table 2. Residual silicone oil levels were determined and are listed in Table 2.

TABLE 2
Residual silicon oil levels in exendin-4 microsphere batches

| | | SiOil | | Residual |
|---|---|---|---|---|
| | | addition | | silicon |
| Lot # | Scale | rate (kg/min) | Addition time (mm:ss) | (ppm) |
| 03-36-166 | 1 kg | 10.1 | 2:13 | 379 |
| 06-002-040 | 1 kg | 0.8; 4.9 | 9:00; 1 min hold; 3:00 | 4.5 |
| SAFC | 15 kg | 14; 60 | 6:00; 5 min hold; 4:00 | <18 |
| 200-75-112 | | (w/nozzle) | | |

The particle size distribution of batch SAFC 200-75-112 obtained as described in Example 1 was as follows: DV50—59.9 μm, DV90—87.9 μm, DV90-DV10—50.7 μm.

### Example 3

### Multi-Stage Coacervation Processes vs. Single Stage Continuous Coacervation Process

### Single Stage Continuous Coacervation Process

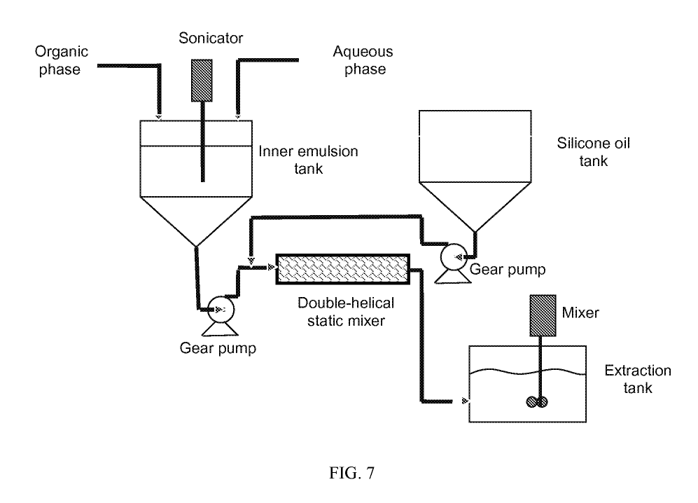

A 6% PLG solution was prepared by dissolving 97.7 g purified 50/50 4A polylactide-co-glycolide (PLG) into 1530 g methylene chloride (DCM). A polypeptide solution was prepared by dissolving 2.1 g sucrose and 5.5 g bovine serum albumin (BSA) into 60 g de-ionized water. The PLG solution (organic phase) was added to the Inner Emulsion tank of the experimental apparatus shown in FIG. 7. The BSA solution (aqueous phase) was added with sonication at 100% amplitude. Sonication was continued for 2 minutes, stopped for 1 minute, and then continued for an additional 2 minutes.

The resulting inner emulsion was combined with 1000 cSt silicone oil and made to flow through a 48 inch double helical static mixer (0.5 inch diameter) at a total flow rate of 12.5 mL/sec (inner emulsion plus silicone oil). The flow rates of the inner emulsion and silicone oil pumps were adjusted to provide a ratio of silicone oil to methylene chloride of 1.0:1.

The outflow of the static mixer was fed into an extraction tank containing 3 L heptane at 3° C. The quench mixture was stirred at 600 rpm for 30 minutes. The hardened microparticles were collected by filtration through a 25 μm sieve, rinsed with cold heptane, vacuum dried and sieved through a 150 μm test sieve. The particle size distribution of microparticles after suspension with sonication in an aqueous diluent containing 3% carboxymethylcellulose, 0.9% NaCl, 0.1% Tween 20 was obtained using a Coulter Multisizer.

### 2 Stage Hybrid Coacervation Process

An inner emulsion comprising BSA and PLG was prepared as described above for the single stage process and then 1000 cSt silicone oil was added with stirring in an amount sufficient to yield a silicone oil to methylene chloride ratio of 0.3:1. The resulting intermediate coacervate was combined with additional silicone oil at the inlet of the 48 inch static mixer. Pump settings were employed to provide a total emulsion flow rate (intermediate coacervate plus additional silicone oil) of 14 mL/sec and a final silicone oil to methylene chloride ratio of 1.0:1. The microparticles were quenched with heptane, collected, dried, sieved and analyzed for particle size distribution as described above for the single stage process.

### 4 Stage Hybrid Coacervation Process

The hybrid coacervation described above was modified to incorporate two additional silicone oil addition stages. 350 cSt silicone oil was added with stirring to the inner emulsion prepared as described above in an amount sufficient to provide a ratio of silicone oil to methylene chloride of 0.5:1. The intermediate coacervate was pumped through three static mixers in series (two 0.5 inch helical static mixers and one Interfacial Surface Generator (ISG) static mixer) with segments of empty pipe to provide residence time between each static mixer. Additional silicone oil was introduced at the entrance to each static mixer. The silicone oil addition rates were selected such that the silicone oil to methylene chloride ratios in the first, second and third static mixers were 0.75:1, 1:1, and 1.5:1, respectively. The empty pipe segments downstream of the first two static mixers were sized to provide residence times of 15 seconds. Microparticles were hardened, collected, dried, sieved and characterized as described above.

TABLE 3
Particle size results for microparticles produced by single
stage continuous coacervation process and hybrid 2 and
4 stage coacervation processes

| | | DV50 | DV90 | Mean | | |
|---|---|---|---|---|---|---|
| | DV10 (μm) | (μm) | (μm) | (μm) | SD (μm) | CV (%) |
| One-stage | 34.1 | 78.4 | 155.3 | 87.5 | 49.8 | 56.9 |
| Two-stage | 25.8 | 48.3 | 90.6 | 54.3 | 26.1 | 48 |
| Four-stage | 27 | 55 | 96 | | | |

### Example 4

Microparticles were prepared using a 2 stage hybrid coacervation process using the experimental setup depicted in FIG. 5. A primary emulsion consisting of 87 grams of an aqueous solution of 3.2% sucrose w/w dispersed in 2170 grams of a 6% w/w PLG solution in methylene chloride. The mixture was chilled and then sonicated for two minutes, allowed to stand for one minute, and then sonicated for an additional two minutes.

Beginning at t=0 min, chilled silicone oil (350 cSt) was added with stirring at 1500 rpm in an amount sufficient to provide the silicone oil to methylene chloride ratio in the intermediate coacervate of 0.5:1 over a period of 5 min 15 sec. A second stage coacervation agent addition was then initiated by pumping the intermediate coacervate through an ISG static mixer with 10 elements (⅜″ diameter; 5″ length), a 300 cm section of ¼ inch ID hollow tubing and a second static mixer of the same type and dimensions at the first. Additional silicone oil was introduced at the entrance of the first static mixer. The intermediate coacervate and silicone oil pumps were set to provide a target final silicone oil to methylene chloride ratio of 1.5:1 and a total coacervate flow rate of approximately 650 g/min.

At t=12 minutes, the flow of intermediate coacervate prior to the silicone oil addition point and static mixer assembly was fed to a stirred quench of 1800 mL heptane and 200 mL ethanol at 3°. At t=13 min a sample of the final coacervate, after the silicone oil addition and static mixer train, was quenched in the same manner.

The intermediate coacervate emulsion was held for an extended period with stirring. The flow of intermediate coacervate and silicone oil into the static mixer assembly was then restarted, and samples of the intermediate coacervate (at t=70 min) and final coacervate (at t=72 min) were quenched as described above.

Material was left in the stirred quench for at least 30 min, collected on 25 um sieves, rinsed with chilled heptane, then dried and characterized. The portion of the final material which passes through a 150 um sieve determines the sieved yield, an indicator of particle size and agglomeration. Microparticles characterized with respect to particle size as described in Example 3. Residual solvent levels were determined using an HP 5890 Series 2 gas chromatograph with an Rtx 1301, 30 m×0.53 mm column. About 130 mg microparticles were dissolved in 10 ml N,N-dimethylformamide. Propyl acetate was used as the internal standard. The sample preparation was adjusted so that concentrations of methylene chloride as low as 0.03% could be quantitated.

Particle size and residual solvent levels are compiled in Table 4. Samples 09-01 and 09-03 are the intermediate coacervates that were quenched without undergoing the second stage of silicone oil addition. Samples 09-02 and 09-04 underwent a second stage silicone oil addition prior to the quench step. The particle size data indicate that no significant particle size growth occurred during the second stage of coacervation, and that the particle size did not increase during the 72 minute intermediate coacervate hold time.

TABLE 4
Particle size and residual solvent results for microparticles
produced by a hybrid 2 stage coacervation process.

| | Sample ID | | | |
|---|---|---|---|---|
| | 09-01 | 09-02 | 09-03 | 09-04 |
| Target ratio (SiOil•MeCl2) | 0.5 | 1.5 | 0.5 | 1.5 |
| Actual ratio | 0.53 | 1.21 | 0.53 | 1.21 |
| Batch hold time (min) | 12 | 13 | 70 | 72 |
| Dv10 (um) | 30.8 | 31.88 | 25.72 | 31.89 |
| Dv50 (um) | 50.76 | 49.59 | 44.31 | 46.99 |
| Dv90 (um) | 84.28 | 81.59 | 80.79 | 82.13 |
| % EtOH | 0.059 | 0.137 | 0.029 | 0.213 |
| % n-heptane | 7.97 | 2.59 | 7.60 | 1.93 |
| Sieved yield | 100% | 100% | 93% | 100% |

### Example 5

Exendin-4 was encapsulated using a 2 stage hybrid coacervation process. An inner emulsion was formed by dispersing an aqueous solution of exendin-4 (5.13 g exendin-4, 1.9 g sucrose in 1381 g water) in a 6% polymer solution (88 g 50/50 4A PLG in 1381 g methylene chloride). Solutions were chilled prior to sonication, and sonication consisted of 2 min sonication, a 1 minute hold time and an additional 2 min sonication. The inner emulsion was added to a chilled reactor (3° C.) and stirred at 1584 rpm (Lightnin Mixer G2S05D, 2″ turbine and 2″ radial flow impellers).

Starting at t=0, chilled silicone oil was added to the stirred reactor over 3 min 27 sec, targeting 690.5 g silicone oil and a silicone oil:methylene chloride ratio of 0.5:1. The intermediate coacervate was combined with additional silicone oil in the apparatus depicted in FIG. 5. The final silicone oil:methylene chloride ratio was 1.65:1 and the flow of coacervate was approximately 475 g/min.

At t=5 min 30 sec, approximately 1 kg of coacervate was fed into a 12 kg heptane:ethanol 90:10 quench. The quench mixture was stirred for 1 hour, allowed to settle for 10 minutes, and then most of the liquid was decanted and 6 kg heptane was added. The mixture was stirred for an additional hour and then fed into a cone dryer, chased with 2.8 kg heptane and dried, yielding sample 200-00042-203A (Table 5).

At t=9 min 10 sec, additional final coacervate was fed into a 2 kg quench of heptane:ethanol 90:10. Flow was suspended and the intermediate coacervate was held until t=60 minutes. The flow of intermediate coacervate and silicone oil was restarted and the resulting coacervate was fed into a second 2 kg quench. The 2 kg quench mixtures were filtered through a 25 um sieve and the resulting microparticles were rinsed with chilled heptane and dried, yielding samples 200-00042-203B and 200-00042-203C.

Microparticle characteristics are compiled in Table 5.

TABLE 5
Particle size, residual solvent, drug load and initial release results
for exendin-4 microparticles produced by a hybrid 2 stage
coacervation process.

| | Sample ID | | |
|---|---|---|---|
| | 200-00042-203A | 200-00042-203B | 200-00042-203C |
| Target ratio | 1.5 | 1.5 | 0.5 |
| (SiOil•MeCl2) | | | |
| Actual ratio | 1.65 | 1.65 | 1.65 |
| Batch hold | 5:30 | 9:10 | 60:00 |
| time (min:sec) | | | |
| Dv10 (um) | 23.8 | 33.8 | 38.0 |
| Dv50 (um) | 40.9 | 52.6 | 81.6 |
| Dv90 (um) | 65.9 | 83.0 | 139 |
| % EtOH | 0.19 | 0.33 | 0.54 |
| % n-heptane | 1.06 | 0.89 | 0.73 |
| % exendin | 4.89 | 4.93 | 4.93 |
| content | | | |
| 1 hr in vitro | 0.98 | 0.31 | 0.23 |
| release (%) | | | |
| Sieved yield | 100% | 100% | 93% |

### Example 6

A microparticle batch was prepared by a continuous coacervation process using a single stage CSTR. An inner emulsion was prepared by dispersing by sonication 65.2 gram of an aqueous solution of a 3.2% aqueous sucrose solution in 1627.5 gram of a 6% solution of PLG (50/50 4A) in methylene chloride. A stirred vessel was charged with 326 gram of the inner emulsion and 451 gram silicone oil (350 cSt) was added over a period of 1 minute. Additional inner emulsion was pumped into the stirred vessel at a rate of 330 g/min, while silicone oil (350 cSt) was added at a rate of 443 g/minute to provide a ratio of silicone oil to methylene chloride of 1.5:1 in the coacervate. Addition of the inner emulsion was sustained for 3 minutes, 34 seconds until exhausted while maintaining a constant volume in the CSTR by gravity feed of coacervate into a quench composed of 22 kg heptane and 2.5 kg ethanol chilled to 3° C. The microparticles were allowed to harden for 60 minutes at 3° C. and then collected, rinsed with heptane, dried under vacuum and sieved.

Microparticles were obtained in a yield of 69%. DV10, DV50 and DV90 values were 12.5, 50.6 and 1114 μm, respectively. Residual ethanol, methylene chloride and heptane levels were 0.31, 0.32 and 2.69%, respectively.

### Example 7

A microparticle batch containing exendin-4 was prepared by a continuous coacervation process using a single stage CSTR. An inner emulsion was prepared by dispersing by sonication a solution of 2.1 gram sucrose and 5.51 gram exendin-4 in 62.9 gram water in 1627.5 gram of a 6% solution of PLG (50/50 4A) in methylene chloride. A stirred vessel was charged with 332 gram of the inner emulsion and 456 gram silicone oil (350 cSt) was added over a period of 1 minute. Additional inner emulsion was pumped into the stirred vessel at a rate of 330 g/min, while silicone oil (350 cSt) was added at a rate of 443 g/minute to provide a ratio of silicone oil to methylene chloride of 1.5:1 in the coacervate. Addition of the inner emulsion was sustained for 3 minutes, 55 seconds until exhausted while maintaining a constant volume in the CSTR by gravity feed of coacervate into a quench composed of 22 kg heptane and 2.5 kg ethanol chilled to 3° C. The microparticles were allowed to harden for 60 minutes at 3° C. and then collected, rinsed with heptane, dried under vacuum and sieved.

A yield of 86.2 grams of exendin-4 microparticles was obtained.

Modifications and variations of the invention will be obvious to those skilled in the art from the foregoing detailed description of the invention. Such modifications and variations are intended to come within the scope of the appended claims.

All patents, patent application publications and articles cited herein are incorporated by reference in their entirety.

1. A method for preparing microparticles comprising
(a) providing a first phase comprising an active agent, a biocompatible polymer and a solvent;
(b) forming a coacervate; and
(c) combining the coacervate with a quench liquid, thereby forming microparticles containing the active agent;
wherein step (b) comprises
(i) adding a first coacervation agent to the first phase; and subsequently
(ii) adding a second coacervation agent.

2. The method of claim 1, wherein the average particle size of the microparticles is between about 1 um and about 250 um.

3. (canceled)

4. The method of claim 1, wherein the active agent is a water soluble drug.

5. The method of claim 1, wherein the active agent is a biologically active polypeptide.

6. (canceled)

7. The method of claim 5, wherein the biologically active polypeptide is a glucoregulatory peptide.

8. (canceled)

9. The method of claim 7, wherein the glucoregulatory peptide is exendin-4.

10. (canceled)

11. The method of claim 5, wherein the polypeptide is present from about 1% w/w to about 5% w/w of the total dry weight of the microparticles.

12-13. (canceled)

14. The method of claim 1, wherein said polymer comprises poly(lactide-co-glycolide).

15. (canceled)

16. The method of claim 14, wherein said polymer has an inherent viscosity of between about 0.3 and 0.5 dL/g.

17. The method of claim 16, wherein the polymer comprises a poly(lactide-co-glycolide) having an uncapped free carboxyl group.

18. (canceled)

19. The method of claim 1, wherein the solvent is methylene chloride.

20. (canceled)

21. The method of claim 1, wherein the first phase is a water-in-oil emulsion comprising the active agent dissolved in the aqueous dispersed phase of the emulsion.

22. (canceled)

23. The method of claim 21, wherein the water-in-oil emulsion has a dispersed phase droplet size of 0.2 μm to 0.4 μm.

24. (canceled)

25. The method of claim 21, wherein the concentration of active agent in the aqueous phase of the water-in-oil emulsion is 50 mg/g to 100 mg/g.

26. The method of claim 1, wherein the first phase is a suspension of particles comprising the active agent in a solution comprising the polymer and solvent.

27. (canceled)

28. The method of claim 26, wherein the average diameter of the suspended particles is less than about 1 μm.

29. The method of claim 1, wherein the first phase comprises the active agent dissolved in the aqueous phase.

30. The method of claim 1, wherein the first phase further comprises an excipient selected from the group consisting of salts, sugars, carbohydrates, buffers and surfactants.

31-33. (canceled)

34. The method of claim 30, wherein the excipient is sucrose.

35. (canceled)

36. The method of claim 30, wherein the excipient and the active agent are dispersed together in the first phase.

37. The method of claim 34, wherein the amount of sugar in the microparticles is 1% to 5% of the total dry weight of the microparticles.

38. The method of claim 37, wherein the amount of sugar in the microparticles is about 2% of the total dry weight of the microparticles.

39. (canceled)

40. The method of claim 30, wherein the total amount of drug and excipient in the microparticles is between 3% and 8% of the total dry weight of the microparticles.

41. (canceled)

42. The method of claim 1, wherein the ratio of polymer to solvent in the first phase is about 20% or less.

43. The method of claim 42, wherein the ratio of polymer to solvent in the first phase is about 10% or less.

44-45. (canceled)

46. The method of claim 1, wherein the first coacervaton agent comprises silicone oil.

47. The method of claim 46, wherein the first coacervation agent and the second coacervation agent are the same.

48. (canceled)

49. The method of claim 1, wherein the weight ratio of coacervation agent to solvent in step (b) is about 0.75:1 to about 2:1.

50. The method of claim 49, wherein the weight ratio of coacervation agent to solvent is about 1.3:1.

51. The method of claim 1, wherein the weight ratio of the first coacervation agent to the solvent is about 0.5:1 or less.

52. The method of claim 1, wherein the first coacervation agent is gradually added to the first phase over a period of at least 3 minutes.

53. The method of claim 1, wherein the quench liquid comprises heptane.

54-56. (canceled)

57. The method of claim 1, wherein the quench liquid comprises a mixture of heptane and ethanol.

58. (canceled)

59. The method of claim 1, wherein step (b) further comprises (iii) adding at least one additional coacervation agent; wherein the additional coacervation agent is the same or different from the first or second coacervation agents.

60. The method of claim 1, wherein step (i) is performed in a stirred tank and the first coacervation agent is added through at least two addition ports.

61. (canceled)

62. The method of claim 60, wherein the first coacervation agent is added through at least one spray nozzle.

63. The method of claim 1, wherein the first coacervation agent and the first phase are made to flow simultaneously through a static mixer.

64. The method of claim 1, wherein the second coacervation agent and a mixture comprising the first phase and the first coacervation agent are made to flow simultaneously through a static mixer.

65. The method of claim 1, wherein the coacervate and a quench liquid are made to flow simultaneously through a static mixer.

66. The method of claim 1, wherein at least one coacervation agent and a mixture comprising the first phase and the first coacervation agent are made to flow simultaneously through a static mixer.

67. A method for preparing microparticles comprising
(a) preparing a first phase comprising an active agent, a biocompatible polymer and a solvent;
(b) forming a coacervate; and
(c) combining the coacervate with a quench liquid, thereby forming microparticles containing the active agent;
wherein step (b) comprises addition of a coacervation agent through at least two inlets of a continuous flow mixing apparatus.

68. The method of claim 67, wherein the continuous flow mixing apparatus is an agitated plug flow reactor with multiple coacervation agent addition ports.

69. The method of claim 67, wherein the continuous flow mixing apparatus is a static mixer assembly with multiple coacervation agent addition ports.

70. The method of claim 67, wherein the continuous flow mixing apparatus is a static mixer with a porous wall through which the coacervation agent is made to flow.

71. (canceled)

72. Microparticles prepared by the method of claim 1.
